(19) United States
(12) Patent Application Publication
(10) Pub. No.: US 2012/0296400 A1
(43) Pub. Date: Nov. 22, 2012

(54) METHOD AND SYSTEM FOR FACILITATING ADJUSTING A CIRCADIAN PACEMAKER

(75) Inventors: Andrew Bierman, Albany, NY (US); Mark S. Rea, Melrose, NY (US); Mariana Figueiro, Troy, NY (US)

(73) Assignee: RENSSELAER POLYTECHNIC INSTITUTE, Troy, NY (US)

(21) Appl. No.: 13/575,788

(22) PCT Filed: Feb. 1, 2011
(86) PCT No.: PCT/US11/23345

§ 371 Date: Jul. 27, 2012

Related U.S. Application Data
(60) Provisional application No. 61/300,072, filed on Feb. 1, 2010.

(51) Int. Cl. A61N 5/06

(52) U.S. Cl. 607/88

(57) ABSTRACT

A method and system for facilitating adjusting a user's circadian pacemaker are provided, including determining a current circadian pacemaker state of the user, as well as potential future states of the user's circadian pacemaker. The potential future states are related to a target circadian pacemaker state representing a circadian pacemaker goal for the user. This is accomplished on a state-variable plane using vectors. An optimum potential future state is selected as a basis for constructing a light exposure treatment schedule for the user. A light exposure treatment schedule is then constructed and provided to the user to facilitate taking control of the user's circadian pacemaker and manipulating it for benefits such as health and performance benefits.

### CROSS-REFERENCE TO RELATED APPLICATION

This application claims the benefit of U.S. provisional patent application Ser. No. 61/300,072, filed Feb. 1, 2010, which is hereby incorporated herein by reference in its entirety.

### STATEMENT REGARDING FEDERALLY SPONSORED RESEARCH AND DEVELOPMENT

This invention was made, in part, with government support under: contract number U01 DA023822 awarded by the National Institutes of Health, National Institute on Drug Abuse (NIDA); contract number R01 OH008171 awarded by the Center for Disease Control (CDC); contract number R01AG034157 awarded by the National Institutes of Health, National Institute on Aging; and contract numbers C080145 and C090145 awarded by the National Science Foundation under Cooperative Agreement EEC-0812056 and the New York State Foundation for Science, Technology and Innovation (NYSTAR). Accordingly, the United States Government may have certain rights in the invention.

### BACKGROUND

All species on the planet, including humans, are exposed to 24-hour patterns of light and darkness as the Earth rotates on its axis. In response to these natural light-dark patterns, species have evolved biological rhythms known as circadian rhythms that repeat approximately every 24 hours. Examples of circadian rhythms include oscillation in core body temperature, hormone secretion, sleep, and alertness. Circadian oscillations occur at the cellular level, including cell mitosis and DNA repair. In mammals, the central circadian pacemaker is located in the suprachiasmatic nuclei (SCN) of the brain's hypothalamus. This master clock provides timing cues throughout the body to regulate the diverse physiological, hormonal and behavioral circadian rhythms. The timing of the circadian pacemaker in humans is slightly longer than 24 hours, so the exogenous light-dark pattern (i.e. natural light-dark pattern caused by the Earth's rotation) resets the timing of the SCN every day as seasons change or as we travel. In this way, our internal clock can be synchronized with the local solar time anywhere on the planet. A breakdown in the synchrony between the circadian pacemaker and the local solar time (as can occur with travel), will disrupt sleep, digestion, alertness, and in chronic cases, research suggests may cause cardiovascular anomalies and/or accelerated cancerous tumor growth.

As an example, epidemiological studies have shown that rotating-shift nurses, who experience a marked lack of synchrony between rest-activity patterns and light-dark cycles, are at higher risk of breast cancer compared to day-shift nurses. More specifically, environmental factors such as electric light at night (LAN) have been implicated as agents in endocrine disruption. It is hypothesized that LAN suppresses pineal melatonin production by the pineal gland, which may shift rest-activity patterns, making them asynchronous with the solar day/night cycle. It has also been shown that melatonin is an antioxidant, significantly retarding the growth of breast cancer and other tumors. In fact, it probably plays a significant role in the development of cancer in mammals. Moreover, in addition to heightened cancer risks, other diseases have been associated with night-shift work, such as diabetes and obesity, which suggests a role of circadian disruption in the development and progression of such diseases.

Though many environmental stimuli have been reported to influence the central circadian pacemaker in mammals, light is established as the dominant environmental stimulus that synchronizes, or entrains, the circadian pacemaker to the local environment, e.g. the light-dark cycle. Furthermore, it is known that light must be incident on the retina to be a stimulus for the human circadian pacemaker. In 2002, a new photoreceptor in the retina was discovered, the intrinsically photosensitive retinal ganglion cell, which has direct nerve projections to the circadian pacemaker in the SCN. This discovery solidified the importance of light in affecting the circadian pacemaker and has invigorated research into light therapy for treating health issues thought to originate from circadian disruption.

The human circadian pacemaker continues to oscillate in the absence of environmental stimuli, but with a free running period slightly different than 24 hrs. In humans, the average free running period is approximately 24.2 hrs. Depending on when light is applied over the course of 24 hrs, it can advance, delay, or have very little effect on the phase of an individual's circadian pacemaker. For instance, light applied before the body reaches its minimum core body temperature will delay the phase of the pacemaker while light applied after the body reaches its minimum core body temperature will advance the phase of the circadian pacemaker. Since the human circadian pacemaker is, on average, slightly longer than 24 hrs, humans generally need morning light to maintain synchronization (or entrainment) between the circadian pacemaker and the local time.

A mathematical model was developed by Kronauer and others that predicts the effect of light on the human circadian pacemaker. The human circadian pacemaker may be modeled as a Van der Pol type limit-cycle oscillator with a nonlinear light dependent driving force. Simulating the behavior of the circadian pacemaker for various light input patterns can be done by numerically solving the set of differential equations that describe the oscillator. However, due to the complexity and nonlinear nature of the model, the reverse operation of solving for a light pattern that achieves a particular desired pacemaker behavior is difficult.

Additionally, up to now, light dosage treatments for circadian pacemaker entrainment have been determined by general guiding principles, such as providing light in the subjective morning to advance the circadian pacemaker or light in the subjective evening to delay the circadian pacemaker, with little timing precision and little or no data to check progress and make adjustments as the treatment proceeds.

### BRIEF SUMMARY OF THE INVENTION

In accordance with an aspect of the present invention, a method of facilitating adjusting a user's circadian pacemaker is provided. The method includes, for instance, constructing a light exposure treatment schedule to facilitate attaining a circadian pacemaker goal for the user, and providing the constructed light exposure treatment schedule to the user to facilitate the user attaining the circadian pacemaker goal. Constructing the light exposure treatment schedule includes, for instance, determining the user's current circadian pacemaker state at a current time t_{c}, ascertaining at least two potential future states of the user's circadian pacemaker based on the user's current circadian pacemaker state, wherein the at least two potential future states are ascertained based on different respective potential light exposure conditions applied to the user, automatically choosing one potential future state of the at least two potential future states for use in constructing the light exposure treatment schedule, the automatically choosing being based on the relation of each potential future state of the at least two potential future states to a target exogenous clock state derived from the circadian pacemaker goal for the user, and constructing the light exposure treatment schedule based on the chosen potential future state.

In another aspect of the present invention, a system for facilitating adjusting a user's circadian pacemaker is provided. The system includes one or more processors to perform constructing a light exposure treatment schedule to facilitate attaining a circadian pacemaker goal for the user, the constructing including determining the user's current circadian pacemaker state at a time t_{c}, ascertaining at least two potential future states of the user's circadian pacemaker based on the user's current circadian pacemaker state, and wherein the at least two potential future states are ascertained based on different respective potential light exposure conditions applied to the user, automatically choosing one potential future state of the at least two potential future states for use in constructing the light exposure treatment schedule, the automatically choosing being based on the relation of each potential future state of the at least two potential future states to a target exogenous clock state derived from the circadian pacemaker goal for the user, and constructing the light exposure treatment schedule based on the chosen potential future state. The one or more processors then perform providing the constructed light exposure treatment schedule to the user to facilitate the user attaining the circadian pacemaker goal.

Additional features and advantages are realized through the techniques of the present invention. Other embodiments and aspects of the invention are described in detail herein and are considered a part of the claimed invention.

### BRIEF DESCRIPTION OF THE SEVERAL VIEWS OF THE DRAWINGS

One or more aspects of the present invention are particularly pointed out and distinctly claimed as examples in the claims at the conclusion of the specification. The foregoing and other objects, features, and advantages of the invention are apparent from the following detailed description taken in conjunction with the accompanying drawings in which:

FIG. 1 illustrates one embodiment of a system for facilitating adjusting a user's circadian pacemaker, in accordance with one or more aspects of the present invention;

FIG. 2 depicts one example of a process for facilitating adjusting a user's circadian pacemaker, in accordance with one or more aspects of the present invention;

FIG. 3 depicts one example of a process for constructing a light exposure treatment schedule for a user, in accordance with one or more aspects of the present invention;

FIG. 4 depicts one example of a state-variable plane used in constructing a light exposure treatment schedule for a user, in accordance with one or more aspects of the present invention;

FIG. 5 depicts an example of a sensing device for facilitating determining a user's current circadian pacemaker state, in accordance with one or more aspects of the present invention;

FIGS. 6A & 6B depict another example of a sensing device for facilitating determining a user's current circadian pacemaker state, in accordance with one or more aspects of the present invention;

FIG. 7 depicts another embodiment of a system for facilitating adjusting a user's circadian pacemaker, in accordance with one or more aspects of the present invention.

### DETAILED DESCRIPTION

The present invention comprises a method that utilizes light exposure and activity data for a user, for instance real-time data, in constructing a light exposure treatment schedule for quickly attaining a circadian pacemaker goal, for instance to recover most quickly from jet-lag, or counteract the disruption of working a night shift.

Generally stated, the present invention comprises a method for using personal light exposure and activity data along with a measure of one's circadian timing to determine light exposure treatment schedules for applying/removing light to/from a user in order to meet desired circadian entrainment goals. Ecological data is collected, such as light exposure data and activity data of a user, and used to estimate circadian timing, for instance a user's circadian pacemaker state. Together with personal light delivery devices, such as LED illuminated glasses, illuminated sleep masks, and specially tinted eyewear, as examples, to remove or apply light when periods of darkness or luminescence are desired, tools are available to affect the circadian pacemaker. This invention bridges the analysis with the treatment to facilitate taking control of one's circadian pacemaker and manipulate it in a systematic fashion for benefits such as health and performance benefits.

Thus, in accordance with an aspect of the present invention, adjusting of a user's circadian pacemaker is facilitated. This includes constructing a light exposure treatment schedule to facilitate attaining a circadian pacemaker goal for the user, and providing the constructed light exposure treatment schedule to the user to facilitate the user attaining the circadian pacemaker goal. In constructing the light exposure treatment schedule the user's current circadian pacemaker state may be determined based on, for instance, obtained data. Then, potential future states of the user's circadian pacemaker are ascertained based on the user's current circadian pacemaker state, and wherein the potential future states are ascertained based on different respective potential light exposure conditions applied. One potential future state of the potential future states is automatically chosen for use in constructing the light exposure treatment schedule, based on the relation of each potential future state of the potential future states to a target derived from the circadian pacemaker goal for the user. Then, the constructed light exposure treatment schedule may be based on the chosen potential future state.

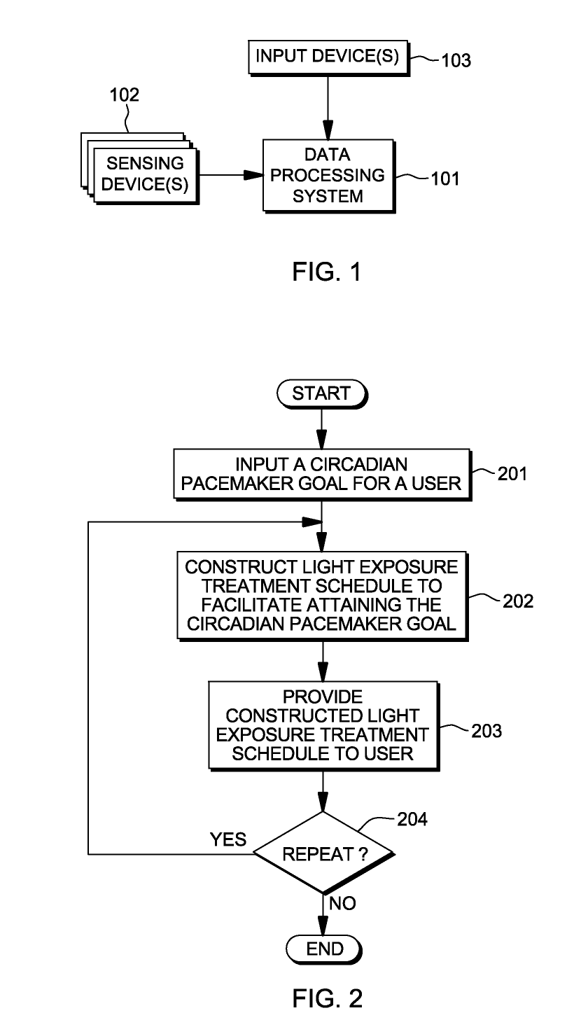

FIG. 1 illustrates one embodiment of a system for facilitating adjusting a user's circadian pacemaker, in accordance with one or more aspects of the present invention. As is shown in FIG. 1, a data processing system 101 is in communication with one or more sensing device(s) 102 and/or one or more input device(s) 103. One or more communications paths 104 may exist between data processing system 101 and sensing device(s) 102, and one or more communications paths 105 may exist between input device(s) 103 and data processing system 101. One non-limiting example of a communications path may comprise one or more digital or analog connections operating via wired or wireless technology to facilitate communication between devices. For instance, a communications path may include a wired connection, an optical connection, and/or a wireless connection. Examples of wireless connections include, but are not limited to, RF connections using a wireless protocol such as an 802.11x protocol, or the Bluetooth® protocol.

Data processing system 101 may include one or more digital or analog components such as data processing units which include one or more processors for performing one or more aspects of the invention described herein. Examples of data processing units which may be used in connection with one or more aspects of the present invention include personal computers (PCs), laptops, workstations, servers, computing terminals, tablet computers, microprocessors, application specific integrated circuits (ASIC), digital components, analog components, or any combination or plurality thereof. Additional examples include mobile devices, for instance personal digital assistants (PDAs) or cellular devices such as smart phones. Additionally, a data processing unit may comprise a stand-alone unit, or it may be a distributed set of devices.

Data processing system 101 may additionally comprise one or more other types of components, such as one or more data storage devices or databases for data logging, storage, and/or retrieval. At least some of the components of data processing system 101 itself may be interconnected by one or more communications paths, such as described above.

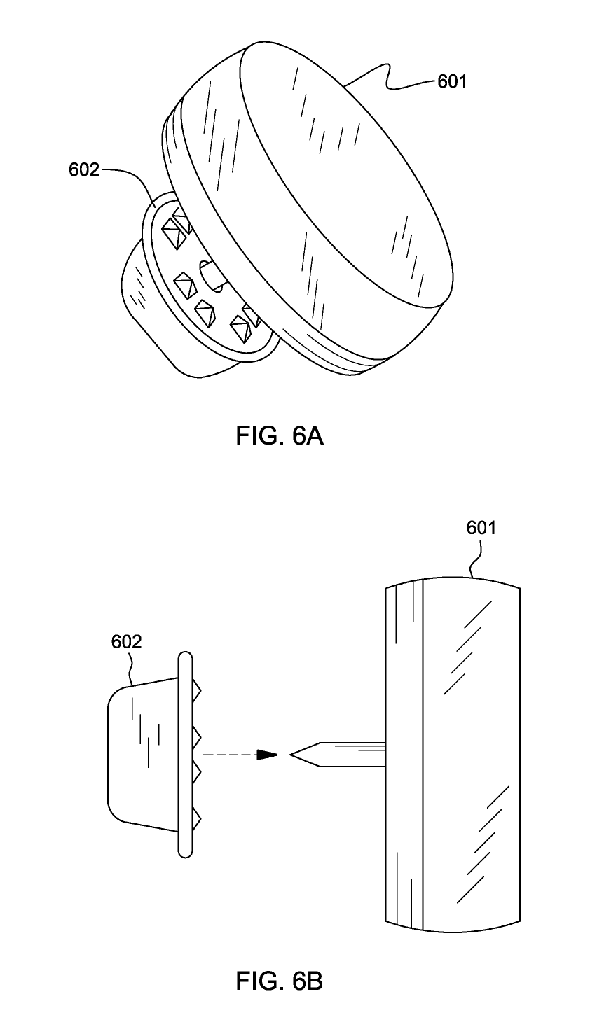

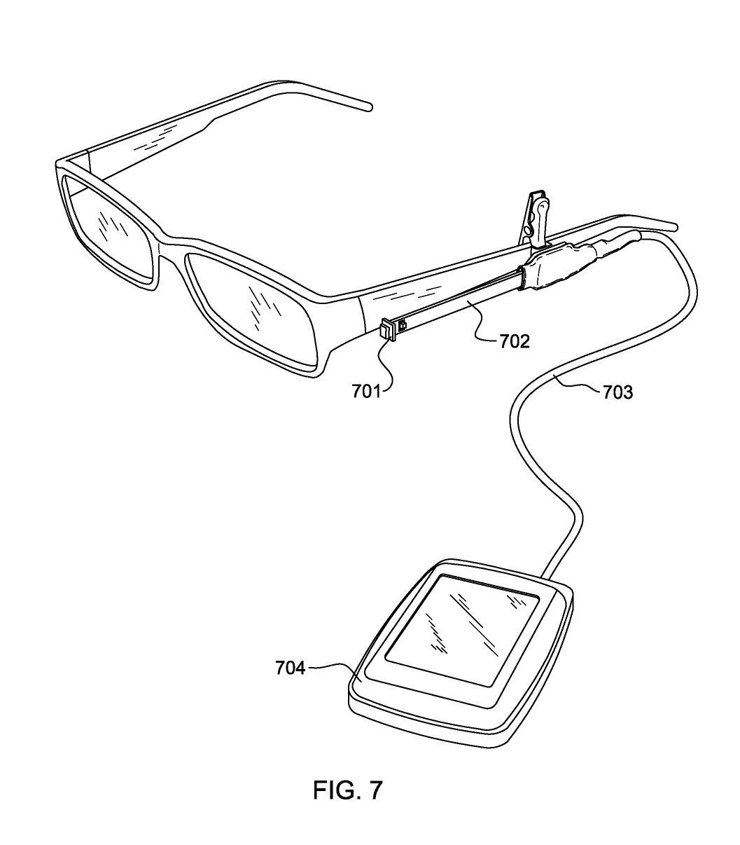

Sensing device(s) 102 of FIG. 1 may be provided for obtaining data, for instance light exposure and/or activity data for a user. Sensing device(s) 102 may be in communication with one or more components of data processing system 101. As one specific example, sensing device(s) 102 may be in communication with a data storage device of data processing system 101, and sensing device(s) 102 may sense ecological and other types of data, which may then be logged either by the sensing device(s) or component(s) of data processing system 101, in the data storage device. Further details and examples of sensing device(s) facilitating one or more aspects of the present invention are described below with reference to FIGS. 5-6.

Continuing with FIG. 1, one or more input devices 103 are provided for facilitating input to and interaction with data processing system 101. Input devices may themselves comprise one or more data processing units, such as a data processing unit as described above. In one embodiment, input device(s) 103 may be provided as one or more components of data processing system 101 itself, or may be provided separate from data processing system 101 and in communication with one or more components thereof (such as depicted in FIG. 1). In one example, input device(s) 103 may facilitate inputting one or more circadian pacemaker goals for a user, as is described below.

Data processing system 101 is configured, in one embodiment, to perform a method for facilitating adjusting a user's circadian pacemaker in accordance with one or more aspects of the present invention. FIG. 2 depicts one example of a process for facilitating adjusting a user's circadian pacemaker, in accordance with an aspect of the present invention. The process begins with input of a circadian pacemaker goal for a user, 201. This input may be accomplished via input device(s) 103 of FIG. 1, as an example. Alternatively or additionally, input may be provided via one or more components of data processing system 101 itself, or may be obtained from a component of the data processing system, such as from a data storage device thereof storing the user's circadian pacemaker goal(s).

Generally, a circadian pacemaker goal may comprise a desired entrainment goal for the user's circadian pacemaker. Inputting the circadian pacemaker goal for the user provides an indication of how the user's circadian pacemaker is to be adjusted. By way of specific example, a user may desire an earlier bedtime, to feel more awake in the morning, to pre-adapt to a different time zone before traveling, and/or to align his or her circadian pacemaker to a particular work-shift schedule or health-treatment schedule, such as a chemotherapy schedule.

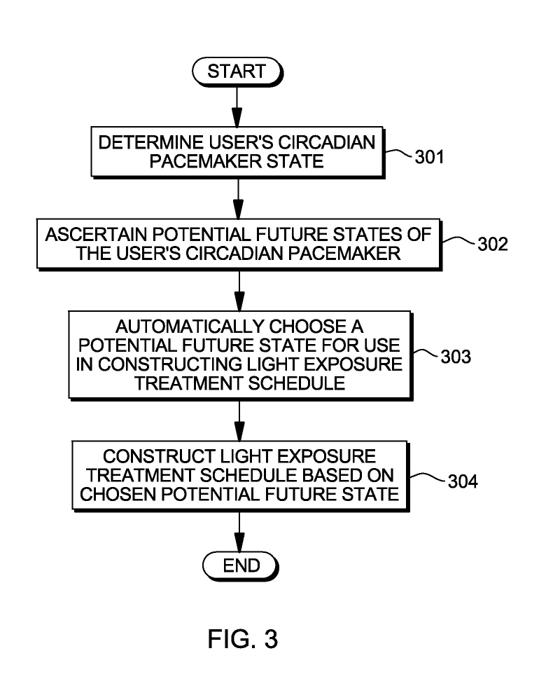

The process in FIG. 2 continues by constructing a light exposure treatment schedule, 202, which is constructed to facilitate attaining the circadian pacemaker goal set for the user. An example of a process for constructing a light exposure treatment schedule for the user is described below with reference to FIG. 3.

Continuing with FIG. 2, after constructing a light exposure treatment schedule, the constructed light exposure treatment schedule may be provided to the user, 203, to facilitate attaining the user's circadian pacemaker goal. Finally, processing determines whether to repeat the constructing and the providing, 204. In one example, this determining occurs automatically after some period of time, and thus the process described herein may dynamically automatically adapt the schedule provided to the user to quickly and efficiently achieve the user's circadian pacemaker goal.

Additionally, or alternatively, a circadian pacemaker goal for the user may be input, updated, or changed at any point during the process depicted in FIG. 2. For instance, a circadian pacemaker goal for the user may be input before, during, or after constructing a light exposure treatment schedule or between repetitions of the constructing and providing, in one example, which enables a circadian pacemaker goal for the user to be readily dynamically adjusted during the process.

Further details regarding construction of a light exposure treatment schedule to facilitate attaining a user's circadian pacemaker goal are described below.

FIG. 3 depicts one example of a process for constructing a light exposure treatment schedule for the user, in accordance with one or more aspects of the present invention. The process begins by determining the user's circadian pacemaker state, 301. In one example, this may be a state of the user's circadian pacemaker at a current time t_{c}, and be based on obtained light exposure data and activity data for the user. A current circadian pacemaker state for a user can be determined by performing a phasor analysis on collected light exposure and activity data to obtain the current state of the user's circadian pacemaker. One example of such an analysis is described in PCT Publication No. WO 2009/073811 A2, published Jun. 11, 2009, which is hereby incorporated herein by reference herein in its entirety. These measures may be useful for diagnosing whether disruption to a user's circadian pacemaker is a likely cause of symptoms and if there would be benefit from light therapy, for example, to improve entrainment or shift the phase of the user's circadian entrainment.

Returning to FIG. 3, after determining the user's circadian pacemaker state, potential future states of the user's circadian pacemaker are ascertained from this state. For instance, potential future states may be ascertained based on different potential light exposure conditions applied to the user, 302. Thereafter, a potential future state for use in constructing a light exposure treatment schedule for the user is automatically chosen, 303. These aspects of the invention are described further below.

The state of a user's circadian pacemaker can be described by two state variables, x and x_{c} that plot on orthogonal axes defining a state-variable plane. A physical interpretation of x may be, for example, the user's core body temperature (CBT), for which the minimum value (CBTmin) is used as a marker for circadian timing.

Additionally, an exogenous clock can also be represented on the same state-variable plane. The exogenous clock corresponds to the circadian pacemaker goal for the user and to which the user's circadian pacemaker is to be, for example, entrained. By way of example, the exogenous clock might represent the 24-hour clock time corresponding to the desired time of CBTmin. The coordinates of the exogenous clock are given by the equation:

x=−cos [(2π/24)*t], x_{c}=sin [(2π/24)*t],

where t is the 24-hour clock time.

The positions of both the exogenous clock and circadian pacemaker change with time, circling about the origin on the state-variable plane. To entrain a user's circadian pacemaker to the exogenous clock, the coordinates of the circadian pacemaker should match those of the exogenous clock, or come to within some predefined close distance. Matching these coordinates ensures the circadian pacemaker has the desired timing (i.e. phase) and amplitude.

As noted, the exogenous clock represents a circadian pacemaker goal for the user. For any time t, there exists some point on the plot of the exogenous clock on the state-variable plane that indicates a target exogenous clock state for that time t. The target exogenous clock state for time t indicates a point in the state-variable plane (and along this exogenous clock) where a user's circadian pacemaker state would be if the user's circadian pacemaker were fully entrained to the exogenous clock (i.e., entrained to the circadian pacemaker goal for the user). Thus, attaining the circadian pacemaker goal for the user comprises aligning the user's circadian pacemaker state for some time t with the state, at that time t, of the exogenous clock to which the user's circadian pacemaker state is being entrained. The pacemaker goal therefore may comprise entraining the user's circadian pacemaker to the exogenous clock.

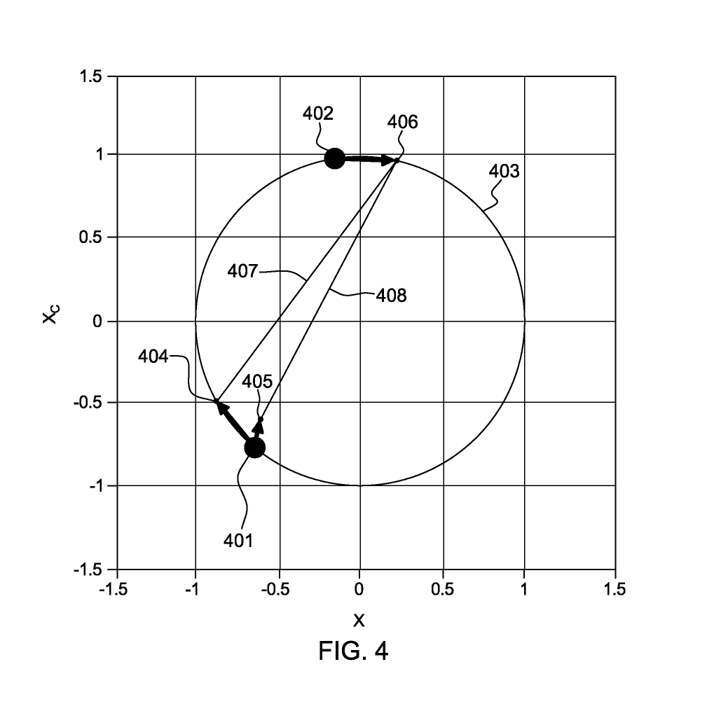

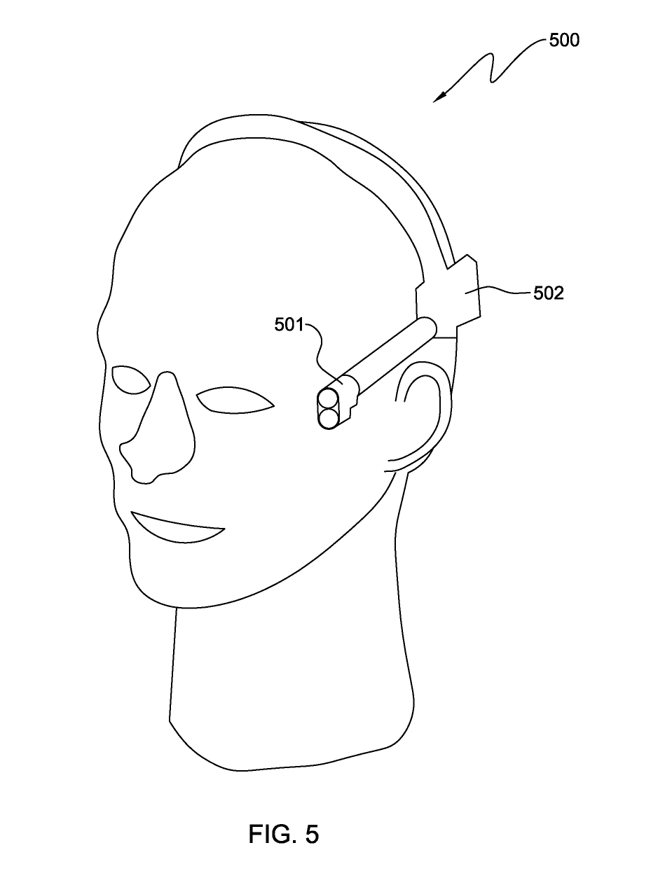

FIG. 4 depicts one example of a state-variable plane used in constructing a light exposure treatment schedule for a user, in accordance with an one or more aspects of the present invention. As noted, a state-variable plane may be used to represent a state of the circadian pacemaker for a user and an exogenous clock which represents the circadian pacemaker goal for the user. The current circadian pacemaker state for the user is identified on the state-variable plane at point 401. Additionally, a current exogenous clock state, point 402, may be identified on exogenous clock 403. Current exogenous clock state 402 on exogenous clock 403 represents the desired circadian pacemaker state for the user at the current time t_{c}. In other words, if the user's circadian pacemaker were fully entrained to the exogenous clock at the current time, points 401 and 402 would align on the state-variable plane. The clockwise distance between points 401 and 402 along exogenous clock 403 is indicative of the offset between the user's current circadian pacemaker and the circadian pacemaker goal for the user.

A state-variable plane may be used in ascertaining potential future states of the current circadian pacemaker state indicated by point 401, for the user. Using phasor analysis for the user (such as that noted above), in conjunction with quantitative model(s) for promoting circadian entrainment, such as is described in Zhang et al., “Circadian System Modeling and Phase Control”, 49^{th} IEEE Conference on Decision and Control, 2010, which is hereby incorporated herein by reference in its entirety, a response of the user's circadian pacemaker to various light exposure conditions may be characterized, quantified and ascertained on the state-variable plane as potential future states of the user's circadian pacemaker for some future time t_{f}. This response is represented on the state-variable plane in FIG. 4 by the magnitude and direction of a vector extending from the current circadian pacemaker state (e.g., point 401). The vector associated with a response to a particular light exposure condition extends to some other point on the state-variable plane, which represents a potential future state of the user's circadian pacemaker for the future time t_{f}, based on the associated, particular light exposure condition. With reference to FIG. 4, points 404 and 405 respectively correspond (by way of example) to ascertained potential future states of the user's circadian pacemaker after a period of time during which no light is applied (point 404), and after a period of time during which illuminance of (for example) 10,000 lux is applied to the user (point 405). The period of time used in ascertaining potential future states may be a consistent amount (for instance 30 minutes, 24 hours, etc.) across each of the potential future states ascertained. It should be noted that point 404 is only coincidentally located along the exogenous clock in the example depicted.

After the potential future states of the circadian pacemaker are ascertained for different light exposure conditions, the process disclosed herein automatically chooses one potential future state for use in constructing the light exposure treatment schedule. This choosing is based on a relation between each potential future state to a target exogenous clock state which, as described above, is a point along the exogenous clock derived from a circadian pacemaker goal for the user at future time t_{f}. As described above, the target exogenous clock state indicates a point in the state-variable plane (and along this exogenous clock) where a user's circadian pacemaker state would be if the user's circadian pacemaker were fully entrained to the exogenous clock.

The target exogenous clock state in FIG. 4 is indicated by point 406 along exogenous clock 403. It indicates the state of the exogenous clock for the future time t_{f}, which was used above in ascertaining the potential future states of the user's circadian pacemaker. The relation between each ascertained potential future state, e.g. point 404 and point 405, of the user's circadian pacemaker and target exogenous clock state 406 may be represented as a vector distance on the state-variable plane, the vector distance extending from the coordinates of the ascertained potential future state to the coordinates of the target exogenous clock state. In FIG. 4, vector 407 extends between point 404 (condition of no light-exposure for the period of time) and target exogenous clock state 406. Likewise, vector 408 extends between point 405 (condition of illuminance of 10,000 lux for the period of time) and target exogenous clock state 406. The vector distances are evaluated and the ascertained potential future state having the shortest vector distance to target exogenous clock state 406 is chosen. An appropriate light exposure treatment schedule is then constructed (FIG. 3 #304) based on the chosen potential future state. For instance, the light exposure condition corresponding to this chosen potential future state may be used to construct the light exposure treatment schedule. In constructing the schedule, other considerations such as input conditions or constraints deemed relevant to the light exposure treatment schedule may be considered and accounted-for in constructed the schedule to be provided to the user, as is discussed below.

As noted, the chosen potential future circadian pacemaker state is the potential future circadian pacemaker state associated with the shortest vector distance to the target exogenous clock state. This guarantees improvement in the entrainment of the user's circadian pacemaker to the exogenous clock (and therefore towards achieving the circadian pacemaker goal), even if the schedule had been previously interrupted or deviated from.

Advantageously, the process of FIG. 3 may be automatically dynamically repeated at some later time t₁, using updated light exposure data and updated activity data. Repeating the process may result in dynamically replacing a prior light exposure treatment schedule with an updated light exposure treatment schedule based on the updated data, and this repeating advantageously results in a schedule of on/off lighting pattern treatments that quickly entrain the circadian pacemaker to the exogenous clock.

An additional benefit of this approach is that it lends itself easily to changing constraints and/or conditions that are important for real-world applications of circadian entrainment, for instance when constructing a light exposure treatment schedule for a user in order to facilitate adjusting the user's circadian pacemaker. Accordingly, changing constraints and/or conditions can be readily accounted for when constructing the updated light exposure treatment schedule.

One example of a constraint may be the availability of times for receiving light treatment. To accommodate a user's schedule, certain periods of the day can be omitted from the light exposure treatment schedule. This may be done by, for instance, simulating the time period for which the constraint applies with the naturally occurring light exposure for that time period. An example of another constraint may be limits on light exposure or intensity available for treatment, including both upper brightness limits and lower darkness limits. Because the calculation process may be repeated any number of times over the duration of minutes, hours, days, weeks, etc., the available intensity level of a light exposure treatment of the light exposure treatment schedule provided may be updated and/or changed during the course of treatment to determine whether available light exposure levels are advantageous or not.

In comparison to the state of the art, methods exist for finding a light pattern solution using a multidimensional unconstrained nonlinear minimization search method (e.g. the Nelder-Mead method), but it is not known whether such an optimization method can make use of measured light exposure data, and not known what its other limitations are. Another potential disadvantage of this prior technique is that there is no guarantee that the light pattern solutions that the method provides are optimal for a given set of conditions. For instance, the method may find it possible to attain entrainment by first driving the user's current circadian pacemaker state toward a state that is not optimal. However, if such an “optimal” light exposure pattern did exist and were used for treatment such as entrainment, an interruption or deviation from the treatment plan could have counterproductive effects that would worsen or delay the entrainment process, making such an optimization process of limited use for practical applications.

In contrast to this, the method disclosed herein always produces a solution that improves the entrainment of the circadian pacemaker at any given time so that even if interruptions or deviations from the constructed light exposure schedule occur in the future, previously applied light exposure(s) are not counterproductive. Furthermore, the present solution is never counterproductive no matter what interruptions or deviations happened in the past.

Further description with respect to the one or more sensing devices 102 (FIG. 1) is provided below with reference to FIGS. 5, 6A & 6B. Sensing device(s) 102 include one or more sensing devices that provide data necessary for quantifying entrainment and establishing the current state of the circadian pacemaker for a user. Some attributes of the sensing device(s) to facilitate this may include, but are not limited to 1) an ability to obtain measurements of a primary stimulus to the circadian system (for instance, light) with measurements of an output marker of the circadian system (for instance, activity, such as indicated by body temperature or melatonin onset), which together enable stimulus-response type analysis; 2) an ability to sense data continuously, for logging thereof over a duration of time, such as multiple days, with reference to a known time standard; 3) an ability to emulate the correct spectral and spatial sensitivity of the human circadian system to light; and/or 4) practicality as a device for the user to wear. This last attribute is in sharp contrast to laboratory methods of measuring circadian phase involving assays on bodily fluids or temperature probes.

One example of sensing device(s) 102 comprises an activity and light-dose sensing device for obtaining light exposure and activity for a user. The obtained data may be provided to the data processing system 101 to facilitate adjusting the user's circadian pacemaker. For instance, this data may be employed in determining the user's current circadian pacemaker state based on the obtained light exposure data and activity data for the user, as described above.

FIG. 5 depicts an example of a sensing device which facilitates one or more aspects of the present invention. Specifically, FIG. 5 depicts an embodiment of an activity and light-dose sensing device 500. Activity and light-dose sensing device 500 measures and characterizes light available for entering a user's eye. The light-dose sensing aspect of device 500 may be configured to measure and characterize a variety of conditions, for example, but not limited to, light intensity, light spectrum, light spatial distribution, and timing/duration of the light. Photosensors 501 for sensing light exposure of a light dose may be mounted substantially at eye-level. Although in the illustrated embodiment photosensors 501 are mounted substantially at eye-level as depicted therein, in other embodiments, the one or more photosensors could be mounted in other locations, such as on a different location of the person, on a work surface, or remote from the person altogether.

Device 500 also includes an activity sensor 502 for recording activity, such as head movements to differentiate between rest/sleep periods and active/awake periods. In other embodiments, the activity sensor could be mounted on a different location of the person or be located remotely from the person as in the case of an activity or motion sensor, such as an infrared motion sensor. Embodiments of activity sensors 502 which are mounted or worn on a person may utilize accelerometers, mercury switches, or the like to sense motion. Depending on the embodiment, activity and light dose sensing device 500 may have one or more light sensors 501 which are coupled to one or more activity sensors 502. In other embodiments, however, one or more light sensors 501 and one or more activity sensors 502 may exist as separate devices. Depending on the embodiment, activity and light dose sensing device 500 may have one or more of signal filtering circuitry, signal processing circuitry, storage circuitry for storing data locally or on a removable storage device, and wired or wireless communication circuitry for transferring stored, buffered, or live data which is collected to a remote device, such as a processor, for analysis or to a remote database for storage. The circuitry may include a data processing system, such as was described above. By way of specific example, the circuitry may include a computer, a microprocessor, an application specific integrated circuit (ASIC), digital electronics, analog electronics, or any combination or plurality thereof.

Activity as measured by device 500 is not a direct measure of the circadian pacemaker in the SCN. Like every downstream measure of circadian function, behavioral activity can only yield partial insight into circadian entrainment. For this reason, the synchrony between light-dark and activity-rest as measured by device 500 might be more precisely operationally defined as “behavioral entrainment.” Since, however, it is presently impossible to directly measure SCN activity, and thus entrainment in the purest sense in living and active humans, the term “entrainment” may describe the observed levels of synchrony between light-dark exposures and activity-rest responses as measured by sensing device(s) 102, such as an activity and light-dose sensing device.

A example device similar to that illustrated in FIG. 5 and which facilitates aspects of the present invention is described in further detail at: Bierman et al., 16 Measurement Science and Technology #11, page 2292 et. seq. (2005), which is hereby incorporated herein by reference in its entirety.

FIGS. 6A & 6B depict another example of a sensing device for facilitating one or more aspects of the present invention. Specifically, FIGS. 6A & 6B provide an alternative embodiment of an activity and light dose sensing device such as depicted in FIG. 5. One form-factor change is to reduce the size of the device while relaxing the requirement of locating the light sensor near the eye. Activity and light dose sensing device 601 may be roughly the size of a dime, with a thickness of about several millimeters. Device 601 may be a self-contained, epoxy encapsulated, battery-powered electronic device that communicates with external equipment, for instance to receive instruction commands and upload logged data, via an optical interface. It may be designed to be worn by the user in a similar fashion as jewelry, or a lapel pin would be worn. For instance, in FIGS. 6A & 6B, device 601 is provided with attachment portion 602, for instance a pin and clasp, for attaching to a shirt collar, lapel, hat, etc. In other embodiments, the device may be fitted with an earring clip or post, or a clip for glasses to facilitate attachment nearer the subject's eyes.

One advantage to device 601 (as compared to, for instance, the device in FIG. 5) is its size and weight, which renders it less cumbersome and more comfortable to wear continuously for extended periods of time. In this regard, making sensing device(s) smaller and less obtrusive reduces the burden on users. This, in turn, is likely to improve subject compliance with wearing the device, permit longer data collection times, and/or provide true continuous data collection including time spent sleeping. Ultimately, this improves effectiveness and efficiency of the adjustment to the user's circadian pacemaker.

An example power source for device 601 comprises a battery to power the device. In one example, the battery is a 3-volt, 55 mA-hr lithium coin cell battery (for example a size CR1616 battery), however a larger battery (for example a size CR2032 battery) may be used depending on the particular environment in which the device is employed and desired battery life.

In one particular embodiment, device 601 may comprise four integrated circuit chips, for instance: 1) a microcontroller unit (such as a MSP430F2274 available from Texas Instruments, Inc., Dallas, Tex.); 2) a digital, 3-axis accelerometer (such as a ADXL345 available from Analog Devices, Inc., Norwood, Mass.); 3) a digital, RGB light sensor (such as a S11059-78HT available from Hamamatsu Photonics K.K.); and 4) a 32-kB serial EEPROM memory chip (such as a 24LC256 available from Microchip Technology, Inc., Chandler, Ariz.). Other electronic components may include resistors, capacitors, a quartz watch crystal (such as a 32 kHz quartz watch crystal) and/or one or more light emitting diodes. The included components may then be mounted on a printed circuit board, for example a fiberglass reinforced epoxy laminate type FR4, and soldered with a tin/lead solder (such as Sn63Pb37).

The electronics and the battery of device 601 may be encapsulated in whole or in part. For instance, they may be encapsulated in whole or in part in a clear casting epoxy (such as Hysol ES 1902 available from Henkel AG & Co. KGaA), and then attachment portion 602 of device 601 may be coupled to device 601 using, for instance, an epoxy or other adhesive, or any other suitable means.

A sensing device, for instance device 601 as described above, may run continuously, but spend a significant portion of time in a low-power sleep mode. In sleep mode, a microcontroller of the sensing device may be shut down, except for an included watch crystal which generates interrupts at discrete time intervals, for instance once per second, to wake up the microcontroller. In an active mode, the microcontroller may operate at a frequency of approximately 1 MHz. When in low-power standby mode, the microcontroller may initiate a light reading after passage of some predefined time, for instance every 5 seconds. If one or more light emitting diodes are provided, they may flash when data is obtained and can be used to verify operation of the device.

In the case where one or more light emitting diodes are provided for the sensing device(s), a light signal (for instance a light signal of a particular color or frequency, such as blue) may be used to command the device(s) to start a new data logging session. The light signal can be simply that from a blue LED if the device is shaded from other ambient light. However, other embodiments may employ a more complicated and/or unique light signal. Logging can be verified by observing one or more LEDs of the sensing device(s) (for example LEDs of a particular color, such as red) flashing at a time interval, for instance of 1 second. In one example, two quick flashes followed by a longer pause may be observed every second. A light signal (for instance a light signal of a particular color or frequency, such as red), delivered in the same fashion as the light signal to command the device(s) to start a data logging session, may command the sensing device(s) to stop the data logging session and return to a low-power standby mode. In one embodiment, logged data is uploaded from the sensing device(s) before starting a new data logging session, for instance in the case when starting a new data logging session will begin to overwrite previously logged data.

A special docking station may be separately provided to upload data to one or more data processing units, for instance data processing units of a data processing system described above with reference to FIG. 1. In one embodiment, once the sensing device is located on the docking station, another light signal (such as a green light signal) may be used to start a data upload procedure.

Accordingly, LED signal source(s) can be used to influence operating modes of the sensing device(s). In one particular embodiment, the docking station may provide necessary light signals to influence the operating mode(s), for instance to start data logging or stop data logging, and to start uploading logged data.

FIG. 7 depicts another embodiment of a system for facilitating adjusting a user's circadian pacemaker, in accordance with one or more aspects of the present invention. In FIG. 7, sensing device(s) 701 may comprise light sensor(s) and be maintained near the user's eyes and, preferably, at the plane of the cornea in order to maintain accuracy for measuring light incident on the eyes of the user. Sensing device(s) 701 may be physically separated from other components (for instance a processor, memory, and/or battery), for instance so that sensing device(s) 701 can advantageously be very small and light weight. A thin stick-shaped sensor board 702 may hold the sensing device(s) 701 at the plane of the eye when the body of the sensor board 702 is attached to the side of the head arm either on eye glasses (for people who normally wear glasses), or to a thin wire headset. Sensor board 702 may be in communication (for instance via a cable 703) with electronics, such as components of the data processing system (FIG. 1 #101), that can be located elsewhere about the body of the user. In this example, sensing device(s) 701 are connected to a smart phone 704 which may serve as the digital processing system (FIG. 1 #101) itself, or as a component thereof. Additionally or alternatively, the additional electronic, such as smart phone 704, may provide activity sensing functionality as described above with reference to activity sensors 502 of FIG. 5.

In one embodiment of a system for facilitating adjusting a user's circadian pacemaker, a smart phone or other device comprising input capability may provide the functionality for a user to input one or more circadian entrainment goals, and upon inputting his or her desired circadian pacemaker goal, the smart phone or device may perform one or more aspects of the present invention to present the user with a constructed light exposure treatment schedule, such as recommendations on when to be exposed to light and when not to. Additionally or alternatively, the smart phone or other device may provide the functionality for a user to input one or more constraints on the constructed light exposure treatment schedule.

Combined with the sensing device(s), which may obtain light and activity data as described above, the smart phone could track how well the person is adhering to the light schedule and remind him or her to avoid light or expose himself or herself to more light, as the case may be, at particular times. In this regard, the smart phone may perform, either individually or in conjunction with one or more other components of the digital processing system, constructing the light exposure treatment schedule and providing the light exposure treatment schedule to the user, for instance on the smart phone's display. One or more aspects of the above may be provided via one or more programs or applications present on the smart phone.

As described above, a light exposure treatment schedule presented to the user via the smart phone is not necessarily static, but rather may continually update, change, and/or be replaced as the data processing system (e.g. the smart phone in the example above) receives updated data from the sensing device(s), and that information is used to re-optimize the light exposure treatment schedule, for meeting the user's circadian entrainment goal. In this way the system is very adaptable to deviations from a set schedule as users goes about their lives. This is important because many circumstances surround a user's light exposure are beyond his or her control. This is why the concept of using updated data obtained from the sensing device(s) and re-optimizing may be important in a practical device. Additionally, the user may be informed of progress toward his or her circadian pacemaker goal, and once the goal is obtained, may be switched to a maintenance mode comprising, for instance, advice on how to stay entrained in line with the circadian pacemaker goal.

Adhering to a light schedule might involve use of personal light delivery devices, such as LED illuminated glasses, illuminated sleep masks, and/or specially tinted eyewear to remove light when periods of darkness are desired. By way of example, avoiding light might involve wearing sunglasses, or filter glasses that block the short-wavelength (blue) part of the spectrum while still allowing light to pass, in order for the visual system to maintain good vision. Exposure to more light at specific times could also be had by wearing glasses with LED sources that aim short-wavelength (blue) light into the eye. Additionally or alternatively, exposure to more light might simply comprise sitting closer to a window, getting outside, or turning on more electric lights. Conversely, avoiding exposure to light may comprise turning off lights or avoiding illuminated areas such as the outdoors or indoor areas in close proximity to windows.

As noted, aspects of the present invention can be used to facilitate adjusting a user's circadian pacemaker. Such adjustment can be useful towards, for instance, helping users improve sleep quality, reducing symptoms of jet lag, promoting earlier bedtimes, and/or reducing risks of diseases, such as cardiovascular disease, diabetes, obesity, and/or cancer. Aspects of the present invention can also be useful to cancer patients undergoing chemotherapy to increase the efficacy of treatment and reduce its side effects. Since humans do not have conscious access to the timing of their circadian pacemaker, the proposed device will serve as a tool to help treat non-pharmacologically those suffering from circadian disruption.

Aspects of the present invention may be used to determine, at any point in time, whether to recommend and/or apply a light stimulus or darkness, in order to facilitate adjusting a user's circadian pacemaker in a short amount of time. Some benefits, as noted above, are that the invention lends itself easily to changing constraints and conditions that are important to include for real-world applications such as people's work and travel schedules and daylight and darkness availability.

As will be appreciated by one skilled in the art, aspects of the present invention may be embodied as a system, method or computer program product. Accordingly, aspects of the present invention may take the form of an entirely hardware embodiment, an entirely software embodiment or an embodiment combining software and hardware aspects. Furthermore, aspects of the present invention may take the form of a computer program product embodied in one or more computer readable medium(s) having computer readable program code embodied thereon.

The computer readable medium may be a computer readable storage medium, such as, for instance, an electronic, magnetic, optical, electromagnetic, infrared or semiconductor system, apparatus, or device, or any combination thereof. More specific examples of the computer readable storage medium include for instance: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any combination thereof. In the context of this document, a computer readable storage medium may be any tangible or non-transitory medium that can contain or store program code for use by or in connection with an instruction execution system, apparatus, or device.

Program code embodied on a computer readable medium may be transmitted using an appropriate medium, including but not limited to wireless, wireline, optical fiber cable, RF, etc., or any combination thereof.

Computer program code for carrying out operations for aspects of the present invention may be written in any combination of one or more programming languages, including an object oriented programming language, such as Java, Smalltalk, C++ or the like, and conventional procedural programming languages, such as the “C” programming language, assembler or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

Although various embodiments are described above, these are only examples. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiment with various modifications as are suited to the particular use contemplated.

1. A method for facilitating adjusting a user's circadian pacemaker, the method comprising:
constructing a light exposure treatment schedule to facilitate attaining a circadian pacemaker goal for the user, the constructing comprising:
determining, by a processor, the user's current circadian pacemaker state at a time t_{c};
ascertaining at least two potential future states of the user's circadian pacemaker based on the user's current circadian pacemaker state, and wherein the at least two potential future states are ascertained based on different respective potential light exposure conditions applied to the user;
automatically choosing one potential future state of the at least two potential future states for use in constructing the light exposure treatment schedule, the automatically choosing being based on the relation of each potential future state of the at least two potential future states to a target exogenous clock state derived from the circadian pacemaker goal for the user; and
constructing the light exposure treatment schedule based on the chosen one potential future state; and
providing the constructed light exposure treatment schedule to the user to facilitate the user attaining the circadian pacemaker goal.

2. The method of claim 1, wherein the target exogenous clock state comprises a state of an exogenous clock, and wherein the circadian pacemaker goal comprises entraining the user's circadian pacemaker to the exogenous clock.

3. The method of claim 2, further comprising plotting the exogenous clock on a state-variable plane.

4. The method of claim 3, wherein the automatically choosing comprises relating the at least two potential future states of the user's circadian pacemaker to the target exogenous clock state on the state-variable plane.

5. The method of claim 4, wherein the relating comprises evaluating vector distance between each potential future state and the target exogenous clock state on the state-variable plane, and wherein the automatically choosing further comprises selecting the potential future state having the shortest vector distance between it and the target exogenous clock state.

6. The method of claim 3, wherein the entraining the user's circadian pacemaker to the exogenous clock comprises matching, within a predefined amount, the user's circadian pacemaker to the exogenous clock on the state-variable plane.

7. The method of claim 1, wherein the at least two potential future states are ascertained for a future time t_{f}. and wherein the target exogenous clock state comprises a state of an exogenous clock for the future time t_{f}.

8. The method of claim 1, wherein the circadian pacemaker goal comprises a desired manipulation to the user's circadian pacemaker.

9. The method of claim 1, wherein the constructed light exposure treatment schedule comprises a first light exposure treatment schedule, and wherein the method further comprises:
automatically repeating the constructing at some later time tₗ to construct a second light exposure treatment schedule, the automatically repeating the constructing at the later time tₗ comprising determining the user's circadian pacemaker state at the later time tₗ; and
dynamically replacing the first light exposure treatment schedule with the second light exposure treatment schedule constructed.

10. The method of claim 9, further comprising updating the circadian pacemaker goal for the user prior to repeating the constructing and the providing the second light exposure treatment schedule.

11. The method of claim 1, further comprising providing by the user one or more constraints on the constructed light exposure treatment schedule, and wherein constructing the light exposure treatment schedule comprises accounting for the one or more constraints.

12. The method of claim 11, wherein the one or more constraints on the constructed light exposure treatment schedule comprise at least one of: availability of a time for receiving a light exposure treatment, or a light exposure limit.

13. The method of claim 1, wherein determining the user's current circadian pacemaker state comprises ascertaining a current level of circadian entrainment of the user, the ascertaining the current level comprising performing phasor analysis on a set of the obtained light exposure data and activity data for the user obtained during a defined interval of time, and wherein the set of obtained light exposure data and activity data comprises measurements of a primary stimulus to the user's circadian pacemaker and measurements of an output marker of the user's circadian pacemaker, and wherein the output marker comprises at least one of minimum core body temperature, or melatonin onset.

14. A system for facilitating adjusting a user's circadian pacemaker, the system comprising:
one or more processors to perform:
constructing a light exposure treatment schedule to facilitate attaining a circadian pacemaker goal for the user, the constructing comprising:
determining the user's current circadian pacemaker state at a time t_{c};
ascertaining at least two potential future states of the user's circadian pacemaker based on the user's current circadian pacemaker state, and wherein the at least two potential future states are ascertained based on different respective potential light exposure conditions applied to the user;
automatically choosing one potential future state of the at least two potential future states for use in constructing the light exposure treatment schedule, the automatically choosing being based on the relation of each potential future state of the at least two potential future states to a target exogenous clock state derived from the circadian pacemaker goal for the user; and
constructing the light exposure treatment schedule based on the chosen one potential future state; and
providing the constructed light exposure treatment schedule to the user to facilitate the user attaining the circadian pacemaker goal.

15. The system of claim 14, wherein the target exogenous clock state comprises a state of an exogenous clock, wherein the circadian pacemaker goal comprises entraining the user's circadian pacemaker to the exogenous clock, and wherein the one or more processors further perform plotting the exogenous clock on a state-variable plane.

16. The system of claim 14, wherein the automatically choosing comprises relating the at least two potential future states of the user's circadian pacemaker to the target exogenous clock state on the state-variable plane, wherein the relating comprises evaluating vector distance between each potential future state and the target exogenous clock state on the state-variable plane, and wherein the automatically choosing further comprises selecting the potential future state having the shortest vector distance between it and the target exogenous clock state.

17. The system of claim 14, wherein the circadian pacemaker goal comprises a desired manipulation to the user's circadian pacemaker.

18. The system of claim 15, wherein the constructed light exposure treatment schedule comprises a first light exposure treatment schedule, and wherein the one or more processors further perform:
automatically repeating the constructing at some later time tₗ to construct a second light exposure treatment schedule, the automatically repeating the constructing at the later time tₗ comprising determining the user's circadian pacemaker state at the later time tₗ; and
dynamically replacing the first light exposure treatment schedule with the second light exposure treatment schedule constructed.

19. The system of claim 14, wherein the user provides one or more constraints on the constructed light exposure treatment schedule, and wherein the constructing the light exposure treatment schedule comprises accounting for the one or more constraints.

20. The system of claim 19, wherein the one or more constraints on the constructed light exposure treatment schedule comprise at least one of: availability of a time for receiving a light exposure treatment, or a light exposure limit.
